(19) United States
(12) Patent Application Publication
(10) Pub. No.: US 2013/0158425 A1
(43) Pub. Date: Jun. 20, 2013

(54) APPARATUS AND METHODS FOR PERFORMING ELECTROTHERAPY

(71) Applicants: Steven Michael Kaye, Malibu, CA (US); Bryan Thomas Kaye, Pacific Palisades, CA (US); Davin Carlton Ashraf, Pacific Palisades, CA (US); Daniel Kemper Walsh, La Jolla, CA (US); John Vito Biondo, Van Nuys, CA (US)

(72) Inventors: Steven Michael Kaye, Malibu, CA (US); Bryan Thomas Kaye, Pacific Palisades, CA (US); Davin Carlton Ashraf, Pacific Palisades, CA (US); Daniel Kemper Walsh, La Jolla, CA (US); John Vito Biondo, Van Nuys, CA (US)

(73) Assignee: Bioelectric Research Corporation, Inc., Carson City, NV (US)

(21) Appl. No.: 13/679,982

(22) Filed: Nov. 16, 2012

Related U.S. Application Data
(60) Provisional application No. 61/561,269, filed on Nov. 18, 2011.
(60) Provisional application No. 61/561,273, filed on Nov. 18, 2011.
(60) Provisional application No. 61/561,275, filed on Nov. 18, 2011.

(51) Int. Cl. A61N 1/04; A61B 5/053; A61N 1/36

(52) U.S. Cl. CPC A61N 1/048; A61N 1/36014; A61B 5/0531
USPC 600/547; 607/115; 607/149; 607/62; 156/60

(57) ABSTRACT

A circumferential electrode includes a moisture-containing layer comprising a first side and an electrically conductive layer on the moisture-containing layer, the electrically conductive layer including a second side and a third side opposite the second side, the third side of the electrically conductive layer contacting the first side of the moisture-containing layer. The circumferential electrode further includes a barrier layer on the electrically conductive layer, the barrier layer including a fourth side adjacent to the second side of the electrically conductive layer. The circumferential electrode further includes a bulk region and a border region, the border region completely surrounding the bulk region, and the electrically conductive layer is in the bulk region but is not in the border region. The circumferential electrode can be part of an electrotherapy system which can be used to apply various current waveforms to patients in order to treat a variety of ailments and conditions.

### CROSS REFERENCE TO RELATED APPLICATIONS

This application claims priority to U.S. Provisional Application Ser. No. 61/561,269, filed on Nov. 18, 2011 and titled “CIRCUMFERENTIAL ELECTRODES FOR ELECTROTHERAPY”, U.S. Provisional Application Ser. No. 61/561,273, filed on Nov. 18, 2011 and titled “CIRCUMFERENTIAL ELECTRODES AND METHODS OF FORMING THE SAME”, and U.S. Provisional Application Ser. No. 61/561,275, filed on Nov. 18, 2011 and titled “APPARATUS AND METHOD FOR PERFORMING ELECTROTHERAPY”, each of which are incorporated herein for all purposes.

### TECHNICAL FIELD

This invention relates to medical devices and methods, specifically devices and methods for performing electrotherapy.

### BACKGROUND

Electrotherapy has been used in a variety of medical and therapeutic healing techniques, for example for treatment of ailments and diseases such as chronic pain, wounds, sprains and strains, lacerations, abrasions, fatigue, Parkinson's disease, diabetes, hemorrhoids, and various others. Clinical trials of electrotherapy systems and techniques for various wound healing applications have resulted in the healing of wounds in a relatively short amount of time, in some cases several weeks, for wounds that did not substantially respond to other conventional forms of treatment applied consistently over the course of a year or longer. In a typical electrotherapy system, a first and a second electrode are each attached to the human body, both electrodes being connected to a current generating source. The current generating source provides an electrical current which passes through the first electrode into the human body, through the portion of the human body between the two electrodes, and back through the second electrode. Optimal current levels and frequency and duration of treatments can depend on a number of factors, including the height, weight, and age of the patient, the relative health of the patient, as well as the ailment or disease being treated.

Currently available electrotherapy systems have a number of shortcomings. The electrodes applied to the patient tend to be bulky and uncomfortable, and in many cases do not consistently provide a sufficiently low resistance contact between the current generating source and the human body. Furthermore, the current generating sources, as well as the electrotherapy systems as a whole, tend to be overly complicated to operate, thereby preventing more widespread adoption of electrotherapy treatments. Hence, improvements in the design and performance of electrotherapy systems are needed in order to enable more widespread adoption.

### SUMMARY

In a first aspect, a circumferential electrode is described. The circumferential electrode includes a moisture-containing layer comprising open-cell foam, the moisture-containing layer having a first side, and an electrically conductive layer on the moisture-containing layer, the electrically conductive layer having a second side and a third side opposite the second side, the third side of the electrically conductive layer being adjacent to the first side of the moisture-containing layer. The circumferential electrode further includes a barrier layer, such as a moisture-proof barrier layer. The moisture-proof barrier layer is on the electrically conductive layer, the moisture-proof barrier layer including a fourth side, the fourth side of the moisture-proof barrier layer being adjacent to the second side of the electrically conductive layer. At least a portion of the open-cell foam has a cell size that is less than 200 microns.

In a second aspect, a circumferential electrode is described. The circumferential electrode includes a moisture-containing layer comprising a first side and an electrically conductive layer on the moisture-containing layer, the electrically conductive layer including a second side and a third side opposite the second side, the third side of the electrically conductive layer contacting the first side of the moisture-containing layer. The circumferential electrode further includes a barrier layer on the electrically conductive layer, the barrier layer including a fourth side adjacent to the second side of the electrically conductive layer.

Circumferential electrodes described herein may include one or more of the following features. The circumferential electrode can comprise a bulk region and a border region, the border region completely surrounding the bulk region, and the electrically conductive layer is in the bulk region but is not in the border region. The barrier layer can be a moisture-proof barrier layer. The circumferential electrode can be configured to be wrapped conformally around a human body part with the moisture-containing layer contacting the human body part. The human body part can be one of an arm, a leg, a neck, or a torso. The circumferential electrode can have a first circumferential length in the absence of a tensile force being applied to the circumferential electrode in a circumferential direction, wherein the circumferential electrode is capable of being stretched to a second circumferential length in the circumferential direction without any structural damage to the circumferential electrode when a first tensile force is applied to the circumferential electrode in the circumferential direction, the second circumferential length being greater than the first circumferential length. The moisture-containing layer can include a fluid, wherein the cell size of the open-cell foam is sufficiently small to prevent substantial leakage of the fluid from one or more edges of the of the moisture-containing layer when the circumferential electrode is wrapped conformally around the human body part and stretched in the circumferential direction to the second circumferential length. The fluid can comprise water, saline, or an electrolyte. The second circumferential length can be at least 1.05 times or at least 1.2 times the first circumferential length.

The circumferential electrode can further comprise a first hook and loop fastening material adjacent to a first edge of the moisture-proof barrier layer and a second hook and loop fastening material adjacent to a second edge of the circumferential electrode moisture-proof barrier layer, the first edge being opposite the second edge, wherein the first and second hook and loop fastening materials serve to secure the circumferential electrode around the human body part. The barrier layer, the moisture-proof barrier layer, the electrically conductive layer, or the moisture-containing layer can be in a shape of a conic section. The cell size of the open-cell foam can be greater than 10 microns, such as between 50 microns and 150 microns. The electrically conductive layer, the moisture-containing layer, the moisture-proof barrier layer, and/or the barrier layer can each have a circumferential length and a lateral length, wherein the circumferential length of the electrically conductive layer is less than the circumferential length of each of the moisture-containing layer and the barrier layer, and the lateral length of the electrically conductive layer is less than the lateral length of each of the moisture-containing layer and the barrier layer. The electrically conductive layer can be positioned between the moisture-containing layer and the barrier layer to define a border region, the border region completely surrounding an outer edge of the electrically conductive layer, wherein the border region comprises a portion of the moisture-containing layer and a portion of the barrier layer but does not include any portion of the electrically conductive layer. The circumferential electrode can further comprise an adhesive layer which attaches the fourth side of the barrier layer to the second side of the electrically conductive layer. The adhesive layer can further attach the portion of the barrier layer in the border region to the portion of the moisture-containing layer in the border region. The adhesive layer can be electrically insulating. The circumferential electrode can be characterized as not requiring an adhesive material between the moisture-containing layer and the electrically conductive layer. The adhesive layer can be elastic or stretchable.

The electrically conductive layer can comprise silver coated cloth or an electrically conductive stretchable adhesive. The barrier layer can comprise nylon-covered closed cell neoprene. The circumferential electrode can further comprise an electrically conductive patch configured to be attached to an output lead, wherein a portion of the barrier layer is between the electrically conductive patch and the electrically conductive layer, and the electrically conductive patch is electrically connected to the electrically conductive layer by a conductive thread sewn through each of the electrically conductive patch, the barrier layer, and the electrically conductive layer. The electrically conductive patch can comprise a first electrically conductive hook and loop fastening material. The circumferential electrode can further comprise an electrically conductive patch configured to be attached to an output lead, wherein the electrically conductive patch directly contacts the electrically conductive layer or is secured to the electrically conductive layer with a conductive adhesive material. The electrically conductive patch can be between the electrically conductive layer and the barrier layer. The circumferential electrode can further comprise an aperture in the barrier layer adjacent to the electrically conductive patch. The electrically conductive patch can comprise a first electrically conductive hook and loop fastening material.

The moisture-containing layer can be electrically conductive. Fluid or moisture in the moisture-containing layer can be electrically conductive. The cell size can be less than 200 microns throughout the open-cell foam. The portion can be a first portion, and the open-cell foam can further comprise a second portion, the first portion surrounding the second portion. The cell size of the open-cell foam in the second portion can be greater than 200 microns. An electrode assembly can be formed which includes any of the circumferential electrodes described herein and the output lead, the output lead comprising a second conductive hook and loop fastening material electrically connected to a lead wire of the output lead, wherein the first conductive hook and loop fastening material is fastened to the second conductive hook and loop fastening material. The assembly can further comprise a current generating source, wherein the output lead is connected to the current generating source.

The circumferential electrode can further comprise an adhesive layer between the electrically conductive layer and the barrier layer, wherein the adhesive layer secures the fourth side of the barrier layer directly to the first side of the moisture-containing layer in the border region, and the adhesive layer secures the fourth side of the barrier layer directly to the second side of the electrically conductive layer in the bulk region. The electrically conductive layer can directly contact the moisture-containing layer without any adhesive material being between the electrically conductive layer and the moisture-containing layer. The third side of the electrically conductive layer can be secured to the first side of the moisture-containing layer with an electrically conductive adhesive material. The border region can have an average width of at least 3 millimeters or at least 6 millimeters. The barrier layer can be configured to prevent or suppress fluid escaping the moisture-containing layer.

The moisture-containing layer can comprise a layer of hydrogel. The layer of hydrogel can include a first portion comprising hygrogel having a first composition, wherein the first composition is configured to adhere to human skin. The first portion can further comprise hydrogel having a second composition, wherein the second composition is configured to adhere to the barrier layer. The first portion can comprise a laminate including the hydrogel having the first composition and the hydrogel having the second composition. The layer of hydrogel can include a second portion comprising hygrogel having a third composition, wherein the third composition is configured to adhere to the electrically conductive layer or to the barrier layer. The second portion can be on an opposite side of the layer of hydrogel from the first portion.

In a third aspect, a method of forming a circumferential electrode is described. The method includes providing a barrier layer comprising a fourth side, and attaching an electrically conductive layer comprising a second side and a third side to the barrier layer, the second side being opposite the third side, the second side being adjacent to the fourth side of the moisture-proof barrier layer. The method further includes adding a hydrogel layer comprising a first side and a fifth side, the first side being opposite the fifth side, the third side of the electrically conductive layer contacting the first side of the hydrogel layer. The hydrogel layer adheres to the barrier layer or to the electrically conductive layer without requiring an additional adhesive.

In a fourth aspect, a method of forming a circumferential electrode is described. The method comprises providing a moisture-proof barrier layer comprising a fourth side, applying an adhesive layer to the fourth side of the moisture-proof barrier layer, and adding an electrically conductive layer comprising a second side and a third side, the second side being opposite the third side, the second side being adjacent to the fourth side of the moisture-proof barrier layer in a bulk region of the circumferential electrode, wherein the adhesive layer is between the electrically conductive layer and the moisture-proof barrier layer. The method further includes adding a moisture-containing layer comprising a first side, the third side of the electrically conductive layer contacting the first side of the moisture-containing layer. The electrically conductive layer is on the moisture-containing layer and the moisture-proof barrier layer is on the electrically-conductive layer, the circumferential electrode comprises a border region completely surrounding the bulk region, the electrically conductive layer is in the bulk region but is not in the border region, the adhesive layer secures the fourth side of the moisture-proof barrier layer directly to the first side of the moisture-containing layer in the border region, and the adhesive layer secures the fourth side of the moisture-proof barrier layer directly to the second side of the electrically conductive layer in the bulk region.

Methods of forming circumferential electrodes can include one or more of the following features. The hydrogel layer can include a first portion adjacent to the first side of the hydrogel layer and a second portion adjacent to the fifth side of the hydrogel layer, the first portion comprising hydrogel having a first composition and the second portion comprising hydrogel having a second composition, wherein the first composition is different from the second composition. The hydrogel in the first portion can be configured to adhere to the barrier layer or to the electrically conductive layer. The hydrogel in the second portion can be configured to adhere to human skin. The hydrogel in the second portion can be further configured to adhere to the barrier layer. The hydrogel in the second portion can comprise a laminate of hydrogel having the second composition and hydrogel having a third composition. The third composition can be the same as the first composition.

The method can further comprise attaching an electrically conductive patch, wherein a portion of the moisture-proof barrier layer is between the electrically conductive patch and the electrically conductive layer, and the electrically conductive patch is electrically connected to the electrically conductive layer by a conductive thread sewn through each of the electrically conductive patch, the moisture-proof barrier layer, and the electrically conductive layer. The electrically conductive patch can comprise a first electrically conductive hook and loop fastening material.

In a fifth aspect, a method of providing an electric current through a recipient is described. The method comprises providing a first electrode and a second electrode, the first and second electrode each contacting the recipient, providing a current generating source connected to each of the first and second electrodes, configuring the current generating source to provide a first electric current in a first direction through the recipient, sensing a first voltage difference between the first electrode and the second electrode to determine a first magnitude of the first voltage difference, comparing the first magnitude to a voltage threshold, and executing a first function. The sensing, the comparing, and the executing are each performed by the current generating source without additional input from an operator or user of the current generating source.

Methods of providing electric currents through a recipient can include one or more of the following features. The recipient can be a human recipient. The sensing and the executing can each be performed at least two times over a time span of at least one second. The executing of the first function can comprise reconfiguring the current generating source to provide a second electric current in the first direction. The first function can be executed when the first magnitude is greater than the voltage threshold. The sensing and the executing can each be performed at least two times over a time span of at least one second, wherein the first magnitude being greater than the voltage threshold comprises the first magnitude exceeding the voltage threshold every time the comparing is performed during the time span. The first function can be executed after a preprogrammed or predefined time span, wherein the first magnitude is less than the voltage threshold when the first function is executed. The second electric current can be smaller than the first electric current.

The method can further comprise executing a second function, the second function being different from the first function, wherein the second function is performed by the current generating source without requiring additional input from an operator or user of the current generating source. The executing of the first function can comprise determining whether the effective resistance between the first and second electrodes is greater than a resistance threshold. The resistance threshold can be at least 200 kilo-ohms. The first function can be performed before the second function. The executing of the second function can comprise providing an alert of an open circuit to a user or operator of the current generating source. The executing of the second function can comprise reconfiguring the current generating source to provide a second electric current in the first direction. The second electric current can be smaller than the first electric current. The first magnitude can be greater than the voltage threshold when the second function is executed.

The method can further comprise sensing a second voltage difference between the first electrode and the second electrode to determine a second magnitude of the second voltage difference, and comparing the second magnitude to the voltage threshold. The sensing of the second voltage difference and the comparing of the second magnitude to the voltage threshold can each be performed by the current generating source without requiring additional input from an operator or user of the current generating source. The method can further comprise reconfiguring the current generating source to provide a third electric current in the first direction. The third electric current can be greater than the second electric current, and the reconfiguring of the current generating source to provide the third electric current can occur a preprogrammed or predefined amount of time after the reconfiguring of the current generating source to provide the second electric current. The third electric current can be less than the second electric current, and the reconfiguring of the current generating source to provide the third electric current can occur a preprogrammed or predefined amount of time after the reconfiguring of the current generating source to provide the second electric current.

In a sixth aspect, an electrotherapy system is described. The electrotherapy system comprises a first electrode and a second electrode, the first and second electrode each being configured to be connected to a biological recipient, and a current generating source connected to each of the first and second electrodes. The current generating source is configured to provide a first electric current in a first direction through the biological recipient, the current generating source comprises means for sensing a first voltage difference between the first electrode and the second electrode to determine a first magnitude of the first voltage difference, means for comparing the first magnitude to a voltage threshold, and means for executing a first function. The current generating source is operable to perform the sensing, the comparing, and the executing without additional input from an operator or user of the current generating source.

In a seventh aspect, a method of performing electrotherapy treatments on a patient is described. The method comprises connecting a current generating source to the patient and configuring the current generating source to provide a first electric current at a first current level setpoint through the patient, the first current level setpoint being between 500 microamps and 5 milliamps. The method further comprises configuring the current generating source to provide a second electric current at a second current level setpoint through the patient, the second current level setpoint being between 10 nanoamps and 1 microamp, passing the first current through the patient for a first time period, wherein the first current has a first mean current value over the entire span of the first time period, and passing the second current through the patient for a second time period, wherein the second current has a second mean current value over the entire span of the second time period. The first current deviates from the first mean current value by less than 10% of the first mean current value throughout the entire first time period, and the second current deviates from the second mean current value by less than 1% of the second mean current value throughout the entire second time period.

In an eighth aspect, a method of performing diagnostic measurements on a patient undergoing electrotherapy is described. The method comprises providing a first electrode and a second electrode, the first and second electrodes each contacting the patient, wherein a portion of the patient's body is between the electrodes. The method further includes providing a current generating source connected to each of the first and second electrodes, the current generating source being configured to provide a current through the patient, passing a first current through the patient for a first time period, the first current being below 200 microamps, raising the current to a second level for a second time period, the second current level being greater than 200 microamps, and reducing the current back to the first current for a third time period, and measuring an impedance during the second time period. The second time period is sufficiently small to prevent substantial changes in the impedance of the portion of the patient's body which is between the electrodes. Furthermore, the second time period can be about 10 milliseconds or less.

### DESCRIPTION OF DRAWINGS

FIG. 1 is an illustrative diagram of an electrotherapy system.

FIG. 2 is a block diagram of a current generating source.

FIG. 3 is a schematic diagram of a current generating source.

FIG. 4 is a perspective view of a circumferential electrode for use in electrotherapy.

FIG. 5A is a cross-sectional view of portions of a circumferential electrode prior to completion of the assembly of the circumferential electrode.

FIG. 5B is a cross-section view of portions of a circumferential electrode prior to completion of the assembly of the circumferential electrode.

FIG. 6 is a plan view of a circumferential electrode.

FIG. 7 is a diagram depicting the shape of a conic section.

FIG. 8 is a cross-sectional view of portions of a circumferential electrode prior to completion of the assembly of the circumferential electrode.

FIGS. 9-10 are illustrations of circumferential electrodes.

Like reference symbols in the various drawings indicate like elements.

### DETAILED DESCRIPTION

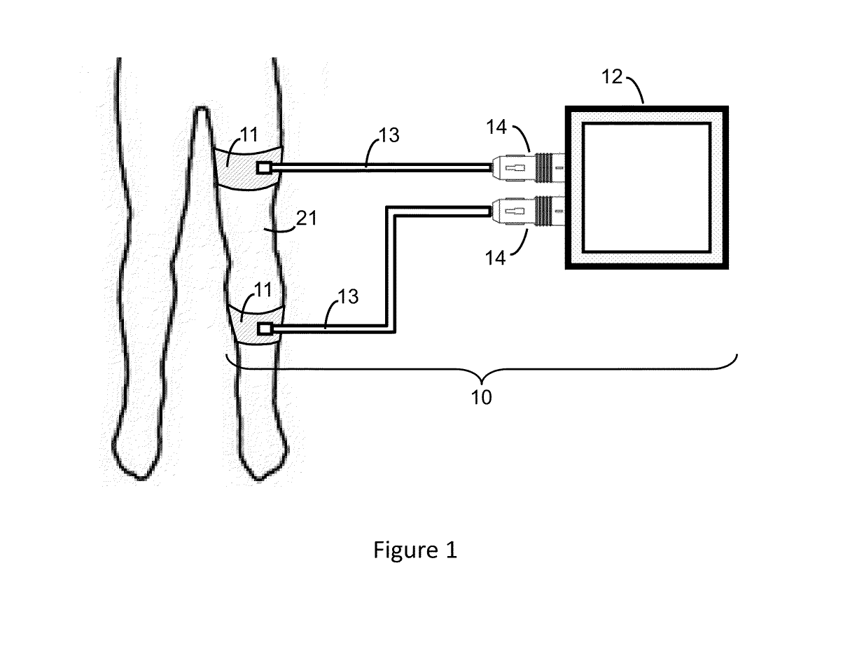

Referring to FIG. 1, a system 10 configured for use in electrotherapy, herein referred to as an “electrotherapy system” 10, includes one or more (typically two) circumferential electrodes 11, a current generating source 12, and output leads 13 which are at one end connected to the circumferential electrode 11 and at the opposite end connected to the current generating source 12. The current generating source 12 is configured to provide an electric current through the portion 21 of the human body that is electrically between the circumferential electrodes 11. The current flows from the current generating source 12 into one of the output leads 13, then the circumferential electrode 11 and into portion 21 of the human body, and then back out through the opposite circumferential electrode 11 and output lead 13. While in FIG. 1, the circumferential electrodes 11 are each shown to be connected to the left leg of the patient, such that the portion 21 through which current flows is a portion of the patient's left leg, the two circumferential electrodes can in general be connected to and encircle any part of the human body. In particular, the electrodes are each typically connected to a patient's leg, arm, neck, head, ankles, or torso. In some implementations, one electrode is connected to each of the patient's ankles. The current generating source can be configured to provide a variety of current waveforms, including DC currents in either direction, sinusoidal waveforms of any period/frequency, stepped waveforms, or any other arbitrary waveform, as well as combinations, for example superpositions or linear combinations, of two or more of the above mentioned waveforms.

In some electrotherapy applications, the voltage applied across the electrodes is maintained at a desired value (or values) by the current/voltage source, with the current adjusting accordingly. In other electrotherapy applications, the amount of current supplied through the electrodes into the human body, rather than the voltage applied across the electrodes, is carefully maintained at a desired value or values throughout the treatment. In the latter type of applications, because the electrical impedance of the portion of the body between the electrodes constantly changes, particularly as current flows, the current generating source 12 is capable of changing the relative voltage between the two circumferential electrodes 11 at a fast enough rate such that the supplied current never deviates from its desired value (i.e., the predetermined or predefined value which the current source was configured/programmed to be supplying) by too large an amount. The amount of deviation that can be tolerated, as well as the length of time for which the deviation occurs, typically depends on the particular application or treatment for which the electrotherapy system 10 is being used. For example, in some applications the maximum deviation from the desired current that can be tolerated is 10% of the desired value, while in other applications it is 5% of the desired value, and in yet other applications it is 1%, 0.5%, or 0.1% of the desired value.

In some applications, larger deviations in current may be tolerable, provided they occur for a very short time, since the body's response time to the supplied currents may be finite. For example, in some implementations, the current does not deviate from its desired value by more than 10% over a given time period, where, depending on the application, the time period may be 1 millisecond, 1 microsecond, or 1 nanosecond. In other applications, the current does not deviate from its desired value by more than 1%, 0.5%, or 0.1% over a time period of 1 millisecond, 1 microsecond, or 1 nanosecond.

The amount that the supplied current deviates from a desired value, as detailed above, is a measurement of the accuracy of the current supplied by the current generating source 12. In some treatments and applications, the precision (rather than the accuracy) of the supplied current is of importance in determining how effective the treatment may be. For example, in some wound healing electrotherapy applications, as long as the current level is somewhat close to (for example, within 25% of) the desired current level, the treatment can enable or expedite healing of the wound as long as the supplied current does not deviate substantially from the mean current. As an example, in cases where the current generating source 12 is configured to supply a constant current for at least 10 consecutive seconds, at least 100 consecutive seconds, or at least 1000 consecutive seconds, the mean (i.e., average) current during treatment may vary from the desired current level (i.e., the predefined or predetermined current level) by less than 20%, less than 15%, less than 10%, less than 5%, or less than 1% of the desired current level, while the current level only deviates from the mean current (or only deviates for a period of time less than 1 millisecond, 1 microsecond, or 1 nanosecond) by less than 5%, 1%, 0.5%, 0.1%, 0.05%, or 0.01% of the mean current value at any time during this time period. When the current is held constant with a high level of precision, physiological responses in the body are thought to enable or enhance the healing process associated with the electrotherapy treatment. In various applications, it has been found that maintaining a current level within 0.1% of the mean current value over a duration of time, such as for at least 10 consecutive seconds, at least 100 consecutive seconds, or at least 1000 consecutive seconds, is crucial to obtaining the desired therapeutic results.

In some electrotherapy applications, accuracy and/or precision of the currents supplied by the current generating source 12 are different at some current levels than those at other current levels. As an example, in some wound healing applications, optimal healing occurs when different constant current levels are each applied for a fixed duration of time. For example, a first constant current in the range of between 500 microamps and 5 milliamps can be supplied for a time period in the range of between 30 seconds and 30 minutes, followed by supply of a second constant current in the range of between 10 nanoamps and 1 microamp for a time period in the range of between 30 seconds and 30 minutes. During application of currents at each of these two current levels, the accuracy and/or precision of the currents necessary to achieve optimal healing from the procedure may be different. For example, at the lower current level, in order to obtain optimal healing effects, the maximum deviation from the desired and/or mean current may be lower at the lower current level than at the higher current level. For example, at the lower current level, the current may deviate from the preprogrammed current level by less than 20% or 10% of the preprogrammed current level and/or from the mean current level by less than 1% or less than 0.1% of the mean current level, whereas at the higher current level, current deviations from the preprogrammed current level as high as 50% of the preprogrammed current level and/or current deviations from the mean current value as high as 10% of the mean current value may be tolerated.

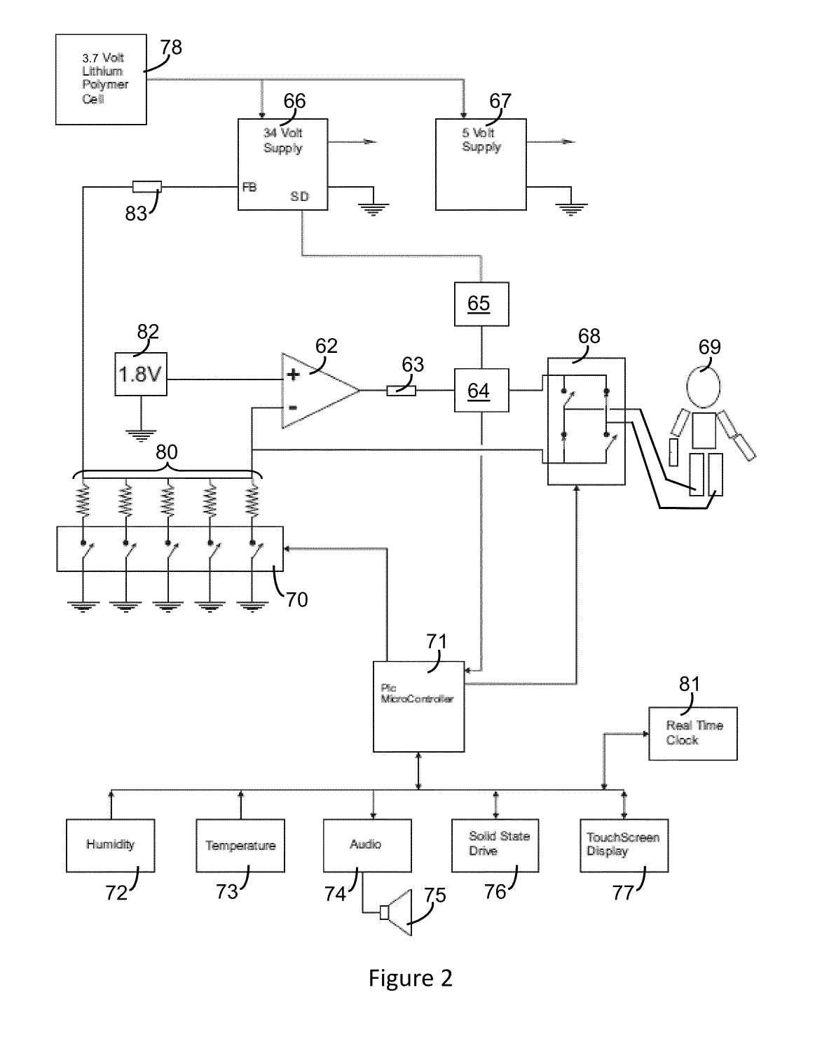

The current generating source 12 can be designed to meet the accuracy and precision specifications described above by employing a feedback loop that can respond to changes in load impedance by changing the relative voltage of the two circumferential electrodes 11 at a sufficiently fast rate to prevent substantial fluctuations in the desired and/or mean current levels. An exemplary block diagram of a current generating source that is suitable for use in the applications described above is shown in FIG. 2. Power for the current generating source is provided by a 3.7 volt lithium polymer cell 78 internal to the case. The LiPo cell powers a 34 volt boost supply 66 and a 5 volt boost supply 67. The 5 volt boost supply 67 provides power for all of the circuitry excluding the analog circuitry supplying current to the patient.

The current to the patient is supplied from the output of an op amp 62. The current flows to the patient through a current limiting diode 63 as an over-current safety measure. Circuit block 64 is a current sensing block which trips latch 65 to disable the 34 volt supply 66 in case of overcurrent. Circuit block 64 also allows the microcontroller 71 to data log the current supplied to the patient, which can be useful for diagnostic purposes. Commutating block 68 allows the polarity of current flow through the patient to be controlled by the microcontroller 71.

Return current from the patient flows through one or more of the parallel-connected resistors of resistor bank 80, the active resistors being selected by analog switch 70. Negative feedback of current through the patient is provided by the voltage dropped across resistor bank 80, and introduced to the inverting input of op amp 62, thereby controlling the current to the patient according to 1.8V/R where R is the parallel resistance controlled by which resistors are activated by analog switch 70. Diode configuration 83 causes any voltage in excess of 4.3 volts at the inverting input of op amp 62 to be applied to the negative feedback terminal of 66. This acts as an additional overcurrent prevention measure. Humidity and temperature inside the case can be read by microcontroller 71 from humidity and temperature sensors 72 and 73, respectively. Audio prompts can be generated by microcontroller 71 and delivered via audio block 74. Solid state drive 76 provides non-volatile storage for data logging. Touchscreen 77 provides user interaction with the system.

As previously described, the current generating source 12 is capable of providing a range of current values, since some applications require lower current levels, others require higher current levels, and still others require various current levels each provided for various time periods. In some implementations, the current generating source 12 is capable of providing currents between 1 femtoamp and 5 milliamps, such as between 1 femtoamp and 1 microamp, between 10 nanoamps and 3 milliamps, or between 100 microamps and 4 milliamps. For example, in various dental applications, a range of approximately 1 femtoamp to 1 microamp has been found to be useful for treating various types of infections. In wound treatment applications, a range of approximately 10 nanoamps to 3 milliamps has proven useful for healing wounds, whereas a range of approximately 100 microamps to 4 milliamps has proven beneficial for treatment of infections associated with the wounds.

Electrotherapy treatments for reduction of withdrawal symptoms for addicts of heroin or other opiates have shown to be effective using an electrotherapy system, such as in FIG. 1, and methods of providing current to the patient as described above. In particular, in outpatient detox treatments, when circumferential electrodes were applied to each of the patient's ankles, and currents in the range of about 200 nanoamps to 200 microamps were applied for a duration of between 20 minutes and 3 hours, withdrawal symptoms were much less severe than those experienced by patients who only took conventional prescription medications. The reduction in pain and other withdrawal symptoms provided by the electrotherapy treatment was sufficient to allow the patients to reduce the amount of prescribed medication necessary for treatment of their symptoms to approximately ⅓ to ½ of the typical dosage while experiencing less pain and/or less severe symptoms than those experienced by similar patients who were provided the full dosage of the prescribed medication without undergoing the electrotherapy treatment.

The maximum voltage differential between the two circumferential electrodes 11 that can be provided by the current generating source 12 is limited by the circuitry within the current generating source 12. In some implementations, the current generating source 12 is only capable of providing voltage differentials in the range of about 0 to 40 Volts or less. As such, if the series resistance of the two circumferential electrodes 11 and the portion 21 of the human body that is between the circumferential electrodes 11 becomes too high during times when the current generating source 12 is configured or programmed to supply a large current, the current generating source 12 may not be capable of providing a sufficiently high voltage to supply the desired current.

The current generating source 12 can therefore be configured to sense the voltage difference between the two electrodes, or alternatively to sense the resistance between the two electrodes. In cases where the series resistance is too large to maintain the desired current level (i.e., the series resistance exceeds a resistance threshold), the voltage difference sensed by the current generating source 12 will exceed a voltage threshold, the voltage threshold typically being close to the maximum voltage differential that the current generating source 12 is capable of providing. If the voltage difference exceeds the voltage threshold (or the series resistance exceeds the resistance threshold) for a sufficiently long time, for example for at least one millisecond, at least one second, or at least three or four seconds, the current generating source 12 can be configured or programmed to automatically step down the desired current level to a predetermined or predefined value without requiring any input from the operator or patient. The current generating source 12 can then resume sensing the voltage difference and/or series resistance between the two electrodes. If the voltage difference is below the voltage threshold (or the series resistance is below the resistance threshold), the setting for the current is maintained. However, if the voltage difference and/or series resistance is still above the voltage or resistance threshold, respectively, the current generating source 12 can again automatically step down the desired current level to a second predetermined or predefined value without requiring any input from the operator or patient. The current generating source 12 can be pre-programmed to continue repeating the above procedure until a current level that can be maintained by the current generating source 12 is found.

Although excessively large resistances through the portion 21 of the patient's body can cause the voltage difference supplied by the current generating source 12 to exceed the voltage threshold, the voltage difference also exceeds the voltage threshold when the circumferential electrodes 11 or output leads 13 are not properly connected, such that there exists an open circuit somewhere in the current path. However, when an open circuit exists, the resistance between the inputs for the output leads 13 is large, typically at least 200 or 250 kilo-ohms, which is much larger than typical resistances when the electrotherapy system 10 is properly connected.

Hence, the current generating source 12 can, for example, be programmed as follows prior to use. First, the current generating source 12 measures or senses the resistance between the inputs that the two output leads 13 are connected to. If the resistance exceeds a resistance threshold, for example 200 or 250 kilo-ohms, the current generating source 12 alerts the user of an open circuit, for example by illuminating an LED, producing a voice warning and/or displaying a message and/or image on a screen. If no open circuit is detected, the internal circuitry of the current generating source 12 is then configured to provide a first electric current, for example about 3 milliamps, in a first direction through the recipient. The current generating source 12 then senses the voltage difference or series resistance between the inputs that the two output leads 13 are connected to, for example sensing the voltage difference or series resistance one time per second or one time per millisecond. If the voltage difference does not exceed the voltage threshold (or the series resistance does not exceed the resistance threshold) for a sustained period of time, for example for a period of between one and ten seconds, the current generating source 12 maintains the current level for a predetermined or predefined amount of time, for example between 1 minute and 30 minutes, after which the current generating source 12 drops the desired current to a second current level, for example about 400 nanoamps, and maintains the second current level for a second predetermined or predefined amount of time, for example between 1 minute and 30 minutes. If the voltage difference does exceed the voltage threshold for a sustained period of time, for example for a period of between one and ten seconds, the current generating source 12 begins successively dropping the desired current level until a current level that can be maintained is found, as described above. Once a sustainable current level is found, that current level is maintained for a predetermined or predefined amount of time, after which the current generating source 12 changes (either increases or decreases) the current to a second desired current level and, if the second current level can be sustained, maintains the current at this second current level for a predetermined or predefined amount of time. In some implementations, a third current level, which may be larger or smaller than the second current level, is then maintained for a third predetermined or predefined amount of time.

While the example above describes current flow in a single direction, any of the treatments can be modified such that for each or any current level maintained by the current generating source 12, the current flows in a first direction for a first predetermined or predefined amount of time, after which the current flows in the opposite direction for a second predetermined or predefined amount of time. In some implementations, the first and second predetermined or predefined amounts of time are the same, while in other implementations they are different.

The current generating source 12 can be configured to perform various diagnostic measurements during treatment. For example, it can include means for measuring and logging parameters about the electrotherapy system 10, the patient, and/or the ambient environment. The parameters can include, for example, resistance between the inputs 15 for the output lead connectors 14, voltage between the inputs 14, reactance (i.e., capacitance and/or inductance) between the inputs 14, current passing through electrodes, temperature, humidity, atmospheric pressure, patient's heart rate, and patient's skin resistance, as well as other parameters. For example, measurements of the resistance, reactance, and/or impedance between the inputs 15 over time (e.g., as a function of time) during operation at a given current level (or levels) can provide certain medical information about the patient, which can be useful in diagnosing certain medical conditions and/or for optimizing conditions for electrotherapy treatments.

During operation at lower current levels, for example below 200 microamps, system noise can cause difficulties in achieving sufficient accuracy in the measurement of one or more of these parameters, for example in measurement of resistance or voltage between the inputs 14. As such, the following method can be employed to achieve a more accurate measurement of these parameters. During operation at a low current level, for example below 200 microamps, the current is raised to a higher current level, for example greater than 200 microamps, for a short time, for example about 10 milliseconds or less or about 1 millisecond or less, and the resistance or voltage measurement is performed during the higher current period of operation, for example at the end of the higher current period of operation. After the resistance or voltage measurement is performed, the current is reduced back to its original level. The duration of time during which high current is supplied (i.e., the high current time) can be less than the time required to cause substantial changes in the impedance of the body between the circumferential electrodes 11. That is, during the high current time, the impedance of the body between the circumferential electrodes 11 can change by less than 2%, less than 1%, less than 0.5%, or less than 0.1%. The higher current level can result in voltage differences between the inputs 14 which are greater than 10 times, greater than 20 times, greater than 50 times, or greater than 100 times the mean voltage fluctuations in the system that result from noise sources such as thermal noise. Higher voltage differences result in more accurate measurements of the parameters, but can also result in larger changes in the patient's impedance. Hence, the current level can be chosen to be the minimum current level that results in a sufficiently accurate measurement of the parameter(s) for the particular application.

In order to simplify use of the electrotherapy system 10 for the operator or user, and to improve functionality, the current generating source 12 can include a variety of functions and features. For example, the current generating source 12 can be programmed to automatically cycle through a set of currents in order to determine the largest current, within the range of currents which the current generating source 12 is configured to output, that can be consistently maintained. The current generating source 12 can further include electrical safeguards, such as current-limiting circuitry, to prevent excessive currents from passing through the patient. The current generating source 12 can also include a touch-sensitive screen for accepting user input and displaying outputs. In some implementations, the current generating source 12 is configured to be able to accept voice commands. To enable a user to better understand error conditions, as well as the proper corrective action an operator could take to eliminate the error, the current generating source 12 can provide voice, alarm, screen, and/or LED warnings, followed by audio and/or visual instructions, step by step, to troubleshoot and correct the error condition(s).

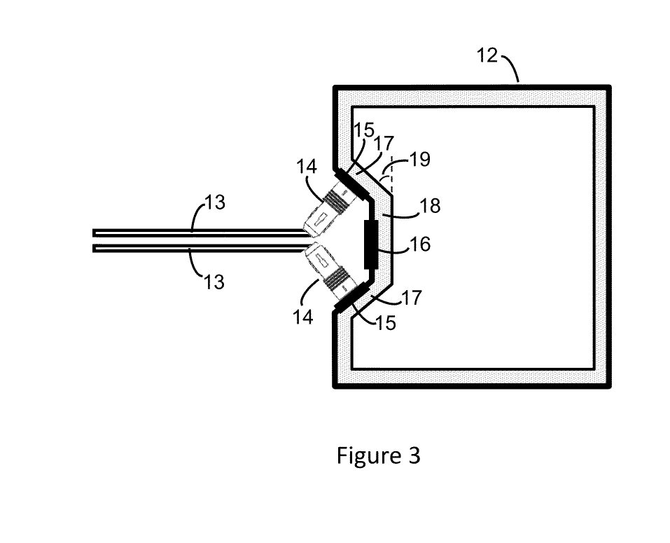

As illustrated in FIG. 3, in order to improve the portability of the electrotherapy system 10, so that the system can be used in locations where an AC wall socket is not present, the current generating source 12 can be battery powered, and can further include a charging input 16 where a charging cable that can be plugged into a wall socket is inserted. However, if the electrotherapy system 10 is operated during times that the current generating source is being charged, in the case of a short circuit or other system failure, the peak voltage supplied at the wall socket, which in some countries can be as high as 350 Volts, can be applied across the two circumferential electrodes 11, thereby resulting in potentially large and harmful currents passing through the patient. In order to prevent such a failure from occurring, the input 16 for the charging cable and the inputs 15 for each of the connectors 14 of the output leads 13 on the current generating source 12 can be positioned such that the charging cable and the output leads cannot physically be simultaneously connected to the current generating source 12. For example, the inputs for the connectors 14 can be positioned such that when the connectors 14 are plugged in to the current generating source 12, the connectors 14 block the input for the charging cable, thereby physically preventing an operator from being able to insert the charging cable into the charging cable input 16.

As seen in FIG. 3, such blocking can be achieve by providing angled portions 17 of the case wall of the current generating source 12, with the inputs 15 for each of the connectors 14 being on the angled portions 17. The portions 17 are angled towards one another, as shown, such that when the connectors 14 are inserted into the inputs 15, they physically block the region where the charging cable is inserted into the charging cable input 16. Such as schematic is desirable in that it allows for use of standard connectors for connectors 14, instead of requiring connectors which are larger than standard. The angle 19 between the angled portions 17 and the portion 18, to which cable input 16 is attached, can be the minimum value required for the connectors 14 to block cable input 16, thereby maximizing space within the case of the current generating source 12, while also allowing for maximum finger access to insert and remove the connector plugs from the inputs/receptacles in the case wall. For example, angle 19 can be 20 degrees or less, such as about 15 degrees or less. In some implementations, portions 17 and 18 of the case wall are each between 0.5 and 0.75 inches, and angle 19 is between 10 and 20 degrees.

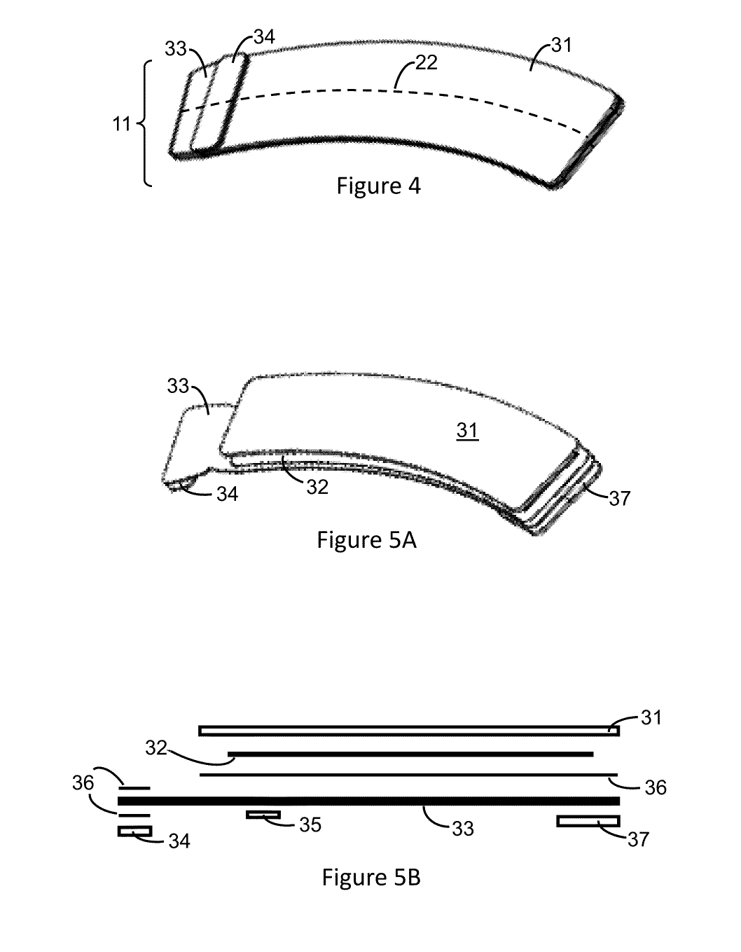

Referring back to FIG. 1, the circumferential electrodes 11 are configured to be wrapped around a body part of the patient, the patient typically being a human patient, although the circumferential electrodes 11 could also be configured for use with other animals. The circumferential electrodes 11 can be configured such that the current supplied by the current generating source 12 passes through the circumferential electrode 11 and into the patient, or from the patient into the circumferential electrode 11, with minimal resistance between the two. For some treatments and applications, it is desirable that the current density (i.e., the current per unit cross-sectional area) passing from the circumferential electrode 11 and into the patient, or from the patient into the circumferential electrode 11, be substantially constant, such that the current is distributed substantially uniformly across the portion of the circumferential electrode 11 which directly contacts the patient. The circumferential electrodes 11 can also be configured to prevent gaps, or other non-ideal features which can impede current flow, between the circumferential electrodes 11 and the body part(s) to which the circumferential electrodes 11 are secured. Finally, the circumferential electrodes 11 can be configured to provide a high degree of ergonomic comfort, and to prevent discomfort caused by effects such as fluid or conducting liquid from leaking from the circumferential electrodes 11 onto the patient, as is common in electrodes used in conventional electrotherapy systems. For example, in many conventional water-based circumferential electrodes, leakage of water or water-based electrolyte is a great problem for comfort, as it results in items of clothing or bedding becoming wet by diffusion upon contact. Other types of electrodes (for example, those that are non-circumferential electrodes and employ hydrogel or other conductive gels) are typically not bulky and may be reasonably comfortable, but do not offer sufficiently uniform current propagation patterns over the cross-section of a circumferential electrode. Descriptions of designs for circumferential electrodes 11 which can achieve these desirable objectives are described below and illustrated in FIGS. 4-10.

FIG. 4 is a perspective view of a circumferential electrode 11, with the inner portion of the electrode (that is, the portion that contacts the patient when the circumferential electrode 11 is attached to the patient) facing upwards. FIGS. 5A and 5B show views of the various components of the circumferential electrode 11 prior to complete assembly of the circumferential electrode.

As illustrated in FIGS. 5A and 5B, a circumferential electrode 11 can include three layers stacked on top of one another. The innermost layer 31 (that is, the layer that contacts the patient when the circumferential electrode 11 is attached to the patient), herein referred to as a “moisture-containing layer” 31, can absorb and retain fluid or moisture, for example after being soaked in a fluid, or contains moisture as a result of the materials and process used to form the layer, such as when the layer 31 is formed of hydrogel. The middle layer 32 is formed of an electrically conductive material, and can be, for example, silver coated cloth or an electrically conductive stretchable adhesive. The outermost layer 33 is a barrier layer, for example a moisture-proof barrier layer. As used herein, a “moisture-proof barrier layer” is a layer which repels fluid or moisture that is incident upon its surface.

The moisture-containing layer 31 forms an electrical contact to the portion of the patient to which the circumferential electrode 11 is attached. That is, electric current supplied by the current generating source 12 is able to pass through the moisture-containing layer 31, and from the moisture-containing layer 31 into the patient with sufficiently low resistance, such that the voltage drop between the portion of the moisture-containing layer 31 adjacent to the patient and a point just below the skin of the patient in the portion covered by the circumferential electrode is minimal, such as less than 1 Volt or less than 0.2 Volts. To form such a contact, the moisture-containing layer 31 can be soaked in an electrically conductive fluid, for example water, preferably water which is non-distilled and/or ionized. Hence, electrical conduction through the moisture-containing layer 31 can occur through the electrically conductive fluid. Or alternatively, an electrically conductive fluid can be embedded in the layer, such as when the moisture-containing layer 31 is formed of hydrogel. Furthermore, the electrically conductive fluid can form a low-resistance, uniform electrical contact to the patient over the entire surface of the moisture-containing layer 31 which contacts the patient.

In order for current to conduct through the fluid in the moisture-containing layer 31, the fluid must be continuous. Hence, the moisture-containing layer 31 can be formed of a material for which absorbed fluid forms a continuous layer. In some implementations, the moisture-containing layer 31 includes or is formed of open-cell foam. The cell size of the open-cell foam can be large enough to allow for a sufficiently conductive fluid layer to be formed within the open-cell foam. For example, the cell size can be greater than 10 microns. As used herein, the “cell size” of open-cell foam refers to the average diameter of each of the cells in the foam. In some implementations, the moisture-containing layer 31 includes or is formed of an electrically conductive material, whereas in other implementations, it is formed of an electrically insulating material.

The moisture-containing layer 31 is capable of retaining the fluid which is absorbed, even while the moisture-containing layer 31 is stretched and/or is under compression, either of which can occur when the circumferential electrode 11 is properly secured to the patient. Fluid retention can be achieved in a number of ways. For example, when open-cell foam is used, the cell size can be sufficiently small, for example less than 300 microns, less than 200 microns, or less than 150 microns, to prevent substantial leakage of the fluid from one or more edges of the of the moisture-containing layer 31 when the circumferential electrode 11 is wrapped conformally and comfortably tight around the patient's body part. Alternatively, the cell size of the open-cell foam can be sufficiently small along the edges or borders of the moisture-containing layer 31, while being larger in the central portion of the moisture-containing layer 31, which can have the advantage of allowing the moisture-containing layer 31 to hold more water or fluid (and thereby be less resistive) while still preventing or suppressing leakage from the edges of the moisture-containing layer 31. When open-cell foam with a sufficiently small cell size, at least along the edges or borders of the moisture-containing layer 31, is used, surface tension effects can prevent or suppress the soaked fluid from leaking out the edge of the moisture-containing layer 31, which can improve the patient's comfort level and improve the useable lifetime of the circumferential electrode 11. Additionally, the area of the moisture-proof barrier layer 33 can be larger than that of the moisture-containing layer 31, such that the moisture-proof barrier layer 31 covers the outer edge of the moisture-containing layer 31 and prevents fluid from leaking out.

In some implementations, the density of cells in the open-cell foam is substantially uniform throughout the open-cell foam, whereas in other implementations it may vary. For example, open-cell foam can be formed for which the cell density varies as a function of thickness. In one implementation, the portion of the open-cell foam close to or adjacent to one of the faces, for example within about 0.25 millimeters of one of the faces, has a first cell density, and the remainder of the open-cell foam has a second cell density which is different from the first cell density. The first cell density can be less than the second cell density, for example about one-half the value of the second cell density, or alternatively be greater than the first cell density. The circumferential electrode 11 can be configured such that the portion of the open-cell foam having a first cell density is distal from the moisture-proof barrier layer 33, and the portion of the open-cell foam having a second cell density is proximal to the moisture-proof barrier layer 33, as in some cases this has been found to improve moisture/fluid retention within the open-cell foam. In other cases, it may be preferable to configure the circumferential electrode 11 such that the portion of the open-cell foam having a second cell density is distal from the moisture-proof barrier layer 33, and the portion of the open-cell foam having a first cell density is proximal to the moisture-proof barrier layer 33.

The moisture-proof barrier layer 33 serves to maintain the fluid within the moisture-containing layer 31 by preventing or suppressing fluid evaporation or wicking away from the moisture-containing layer 31. As such, the moisture-proof barrier layer 33 can cover at least 90% of the area of the moisture-containing layer 31. In some implementations, the moisture-proof barrier layer 33 covers the entire outer area of the moisture-containing layer 31 (that is, the area of the surface of the moisture-containing layer which is opposite the patient's skin). In some implementations, the moisture-proof barrier layer 33 includes or is formed of nylon-covered closed cell neoprene.

As previously described, in many types of electrotherapy treatments, it is preferable that the current passing from the circumferential electrode 11 into the patient's body, or vice-versa, be uniformly distributed across the surface of the circumferential electrode 11 which contacts the patient. Simulations utilizing the transmission line modeling (TLM) method indicate that optimal distribution of the current can occur in the circumferential electrode 11 if the electrically conductive layer 32 is highly conductive, and the moisture-containing layer 31 is moderately conductive. That is, the electrically conductive layer 32 has a higher electrical conductance in the direction of current flow than the moisture-containing layer 31, for example at least 2 times, at least 10 times, or at least 50 times the electrical conductance of the moisture-containing layer 31. The resistivity of the moisture-containing layer 31 can be optimized to achieve uniform current distribution without through the circumferential electrode 11 without adding too much series resistance. That is, if the resistivity is too small, the current passing from the circumferential electrode 11 into the patient may not be distributed uniformly over the surface of the electrode which contacts the patient. On the other hand, if the resistivity is too high, then the series resistance in the circuit may become too large to supply the desired current levels to the patient. Resistivities in the range of about 3-30 kiloOhm-cm, for example between 10 and 20 kiloOhm-cm, have been found to allow for sufficient current spreading without adding too much series resistance.

Alternatively, the circumferential electrode 11 in FIGS. 4-5 can be modified as follows and still achieve substantially uniform current distribution over the surface of the electrode. The moisture-containing layer 31 can be highly conductive, but form a large interface resistance between the patient and the portion of the electrode contacting the patient. In this case, the electrically conductive layer 32 could be eliminated.

The circumferential electrode 11 can include additional features which allow the surface contacting the patient's body to better conform to the patient's body part, thereby improving the electrical contact between the circumferential electrode 11 and the patient, as well as improving the comfort of the circumferential electrode 11 for the patient. In some implementations, the circumferential electrode 11 is stretchable. That is, when a tensile force is applied along the length of the circumferential electrode 11, the circumferential electrode 11 can be capable of being stretched without causing damage to the circumferential electrode 11. For example, consider a circumferential electrode 11 having a first circumferential length when no tensile force is applied, where the circumferential length is defined as the length along the center of the electrode, i.e., the length of dashed line 22 in FIG. 4. When a tensile force is applied to the circumferential electrode 11 in a circumferential direction, i.e., along the direction of the circumferential length, the circumferential length can increase to at least 1.05 times, 1.1 times, 1.15 times, or 1.2 times the first circumferential length without causing structural damage to the circumferential electrode 11. To allow for a minimal number of electrodes with different circumferential lengths to be able to be used with at least 95% of the population in the United States, an increase in the circumferential length to at least 1.1 times the unstretched length is desirable, although an increase of at least 1.2 or 1.3 times the unstretched length may be preferable. In this case, when open-cell foam is used for the moisture-containing layer 31, the cell size can be sufficiently small, at least along the perimeter of moisture-containing layer 31, to prevent or suppress leakage of the fluid in the moisture-containing layer 31 from one or more edges of the moisture-containing layer 31 when the circumferential electrode is wrapped conformally around the human body part and stretched in the circumferential direction. For example, the cell size can be less than 200 microns or less than 150 microns.

The conformal fit of the circumferential electrode 11 to the patient's body part can further be improved by forming the circumferential electrode 11 in the shape of a conic section, for example by forming the moisture-proof barrier layer 33, the electrically conductive layer 32, and/or the moisture-containing layer 31 in the shape of a conic section. As shown in FIG. 7, a conic section shape of a material can be obtained as follows. A layer of the material is formed in the shape of a surface of a cone having an angle θ, as shown. A portion 41 is then removed from the cone, the portion 41 being in the shape of a conic section. The portion 41 includes an outer circumference 42, and inner circumference 43 which is smaller than the outer circumference 42, and a middle circumference 44, where the length of the middle circumference can define at least in part the circumferential length of the circumferential electrode 11. Although in FIG. 7 the portion 41 is shown to have a lateral length of 3 inches, corresponding to a circumferential electrode 11 having a lateral length of about 3 inches, other lateral lengths, for example 2 inches or 1-5 inches, can be used as well.

The conical shape of the circumferential electrode 11 can improve the conformal fit of the circumferential electrode 11 over the patient's body part and can help prevent gaps or bubbles between the circumferential electrode 11 and the patient's body which result in regions through which current cannot pass from the circumferential electrode 11 to the patient. As an example, when a circumferential electrode 11 is fastened around the leg of the patient, the outer circumference 42 of the electrode is positioned higher up on the leg, closer to the patient's torso, and the inner circumference 43 is positioned lower down on the leg, closer to the patient's foot. The angle θ of the cone defining the conic section shape of the circumferential electrode 11 can be optimized to provide a conformal fit to different sized patients. In some implementations, the angle θ is between 1 and 15 degrees, such as between 2 and 10 degrees or between 3 and 7 degrees. In other implementations, the angle θ is about 5 degrees.

The circumferential electrode 11 can further include means for securing the circumferential electrode 11 around the patient's body part. By way of example, as illustrated in FIGS. 4-6 and 8-9, the securing means can include hook and loop or Velcro brand fastening material. Referring to FIG. 4, a first hook and loop fastening material 34 is adjacent to one end of the circumferential electrode 11. The first hook and loop fastening material 34 is positioned on the inner portion of the circumferential electrode 11, i.e., the portion of the circumferential electrode adjacent to the body part around which the circumferential electrode 11 is secured. A second hook and loop fastening material 37, not shown in FIG. 4 but illustrated in FIGS. 5A and 5B, is adjacent to the opposite end of the circumferential electrode 11 from the first hook and loop fastening material 34. The second hook and loop fastening material 37 is positioned on the outer portion of the circumferential electrode 11, i.e., the portion of the circumferential electrode distal from the body part around which the circumferential electrode 11 is secured. In order to secure the circumferential electrode 11 around a body part of a patient, the circumferential electrode 11 is wrapped around the body part, and the first and second hook and loop fastening materials 34 and 37, respectively, are fastened to one another.

In implementations where the moisture-containing layer 31 is formed of hydrogel (or another conductive gel), the hydrogel may be self-adhesive to the patient's skin, at least in regions where the patient does not have too much hair. As such, in these implementations, additional means for securing the circumferential electrode to the patient may be unnecessary. Instead, the circumferential electrode can be made to have a larger circumference than is required for most or all patients, and prior to use the patient can cut the electrode to an optimal circumferential length. In cases where self-adhesion of the hydrogel to the patient is not possible, for example if the patient has too much hair in the region to which the electrode is to be attached, a snap bracelet mechanism can be employed on the outermost portion of the circumferential electrode so that a first end of the electrode can be wrapped over the opposite end and will adhere to the opposite end. More specifically, referring to FIG. 5B, when layer 31 is formed of hydrogel, layers 34 and 37 can be eliminated, and layer 33 can be formed of a material to which the hydrogel can adhere, such that the hydrogel 31 adheres to layer 33 when the electrode is wrapped around the patient with one end extending over the outer portion of layer 33. The end of the electrode that is wrapped over layer 33 can include a snap bracelet, or other means by which the patient or health provider can easily grip the end in order to facilitate removal of the electrode from the patient.

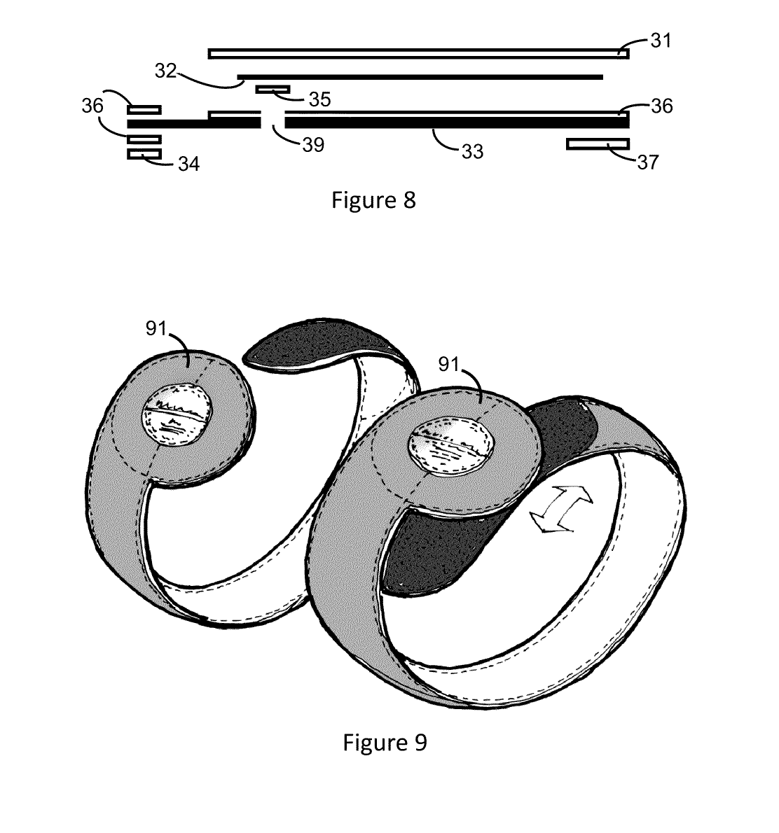

The circumferential electrode 11 further includes a means for electrically connecting the output lead 13 to the electrically conductive layer 32 of the circumferential electrode 11, herein an “electrically conductive patch”. The electrically conductive patch 35, shown in FIG. 5B, is a conductive material, for example conductive hook and loop material, that is in electrical contact with the electrically conductive layer 32. In some implementations, the moisture-proof barrier layer 33 is electrically conductive or includes an electrically conductive portion contacting the electrically conductive layer 32, in which case the electrically conductive patch 35 is connected directly to the electrically conductive portion of the moisture-proof barrier layer 33. However, in other implementations, the moisture-proof barrier layer 33 is formed of an electrically insulating material, in which case the electrically conductive patch 35 can be electrically connected directly to the electrically conductive layer 32 while still being on the outer portion of the circumferential electrode 11, i.e., on an opposite side of the moisture-proof barrier layer 33 from the electrically conductive layer 32. This can be achieved by sewing the electrically conductive patch 35 to the circumferential electrode 11 using an electrically conductive thread, where the thread is stitched through the electrically conductive patch 35, the moisture-proof barrier layer 33, and the electrically conductive layer 32. Alternatively, as further described below and in FIG. 8, the electrically conductive patch 35 can be between the moisture-proof barrier layer 33 and the electrically conductive layer 32, with the electrically conductive patch 35 directly contacting or conductively bonded to the electrically conductive layer 32. In this case, an aperture can be formed in the moisture-proof barrier layer 33 and the adhesive layer 36 which exposes at least a portion of the electrically conductive patch 35.

The output lead 13 at one end includes a means for connecting to the electrically conductive patch 35. For example, when the electrically conductive patch 35 is formed of a first hook and loop fastening material, the output lead can include a second hook and loop fastening material configured to be connected to the first hook and loop fastening material, where the second hook and loop fastening material is connected to the conductive wire of the output lead 13.

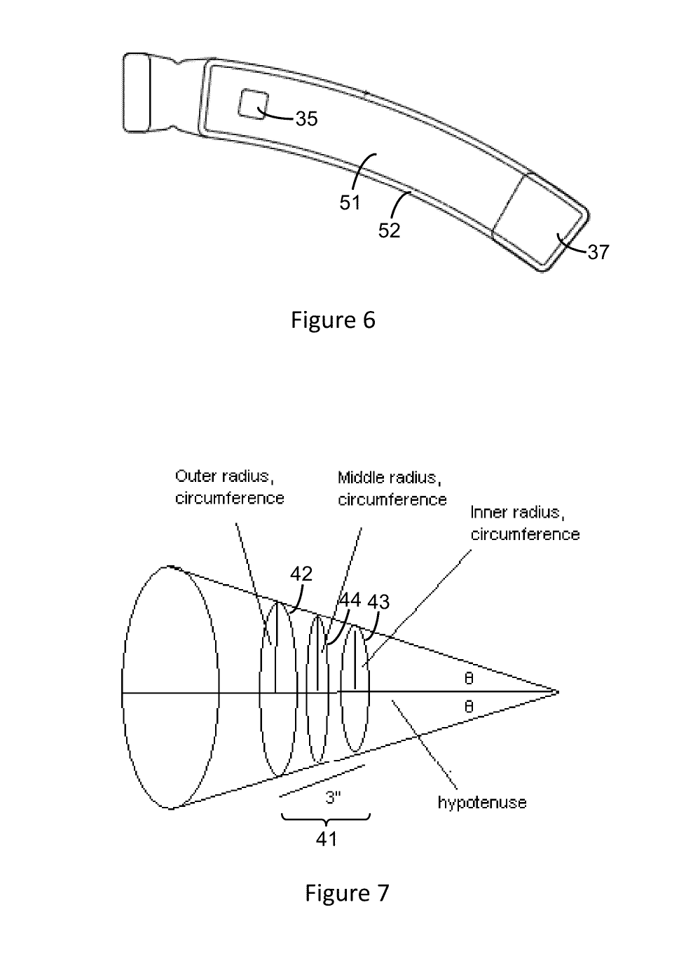

A method of assembling a circumferential electrode 11 is as follows. Referring to FIG. 5B, the electrically conductive layer 32 is first secured to the moisture-proof barrier layer 33 using an adhesive layer 36, where the adhesive layer 36 is typically electrically insulating but may be electrically conductive. As seen in FIGS. 5B and 6, the area of the electrically conductive layer 32 is smaller than that of both the moisture-proof barrier layer 33 and the adhesive layer 36, such that the entire area of the electrically conductive layer 32 is attached to the moisture-proof barrier layer 33 in a bulk region 51 of the circumferential electrode 11, and a border region 52 which includes only the moisture-proof barrier layer 33 and the adhesive layer 36 surrounds the bulk region 51. The electrically conductive patch 35 is then sewn onto the circumferential electrode 11 using an electrically conductive thread, where the thread is stitched through the electrically conductive patch 35, the moisture-proof barrier layer 33, the adhesive layer 36, and the electrically conductive layer 32. Next, the moisture-containing layer 31 is attached. The area of the moisture-containing layer 31 is larger than that of the electrically conductive layer 32, such that the moisture-containing layer 31 is in both the bulk and border regions. Hence, the moisture-containing layer 31 is secured to the moisture-proof barrier layer 33 by the adhesive layer 36 in the border region 52, while contacting the electrically conductive layer 32 in the bulk region 51 without any of the adhesive material being between the moisture-containing layer 31 and the electrically conductive layer 32. The border region can be wide enough to secure the layers to one another without the layers subsequently becoming detached. For example, the border region can have an average width of at least 2 mm, at least 3 mm, or at least 6 mm. Having a single adhesive layer 36, rather than also including a second adhesive layer between the moisture-containing layer 31 and the electrically conductive layer 32, simplifies the manufacturing process of the circumferential electrode 11, as well as eliminating the need for electrically conductive adhesive material within the circumferential electrode, which can be advantageous since in many cases electrically insulating adhesives may exhibit superior adhesion properties as compared to electrically conductive adhesives.

After attaching the moisture-containing layer 31, the first and second hook and loop fastening materials 34 and 37, respectively, are attached to the circumferential electrode 11 on the outer surface of the moisture-proof barrier layer 33, as shown in FIG. 5B, resulting in the partially completed circumferential electrode illustrated in FIG. 5A. As seen in FIG. 5B, the first hook and loop fastening material 34 is attached to a flap on the moisture-proof barrier layer 33 which does not have either of the moisture-containing layer 31 or the electrically conductive layer 32 on the opposite side. Adhesive material 36 is further included on the flap on the opposite side of the moisture-proof barrier layer 33 from the first hook and loop fastening material 34. The flap is then folded over, such that the adhesive material on the flap secures the flap to the inner portion of the circumferential electrode 11, resulting in the circumferential electrode 11 shown in FIG. 4. Although FIG. 4 shows a portion of moisture-proof barrier layer material 33 between the first hook and loop fastening material 34 and the edge of the circumferential electrode 11, the first hook and loop fastening material 34 can extend all the way to the edge. Additionally, the second hook and loop fastening material 37 can be longer and extend further towards the first hook and loop fastening material 34, as compared to the illustration shown in FIG. 5B, in order to allow for a larger range of limb circumferences to be accommodated by a single length circumferential electrode.

An alternative method of assembling a circumferential electrode is as follows. Rather than employing an adhesive layer to secure the electrically conductive layer 32 to the moisture-proof barrier layer 33, the electrically conductive layer 32 can be formed of an electrically conductive vapor which is sprayed directly onto the surface of the moisture-proof barrier layer 33 without an adhesive layer between the electrically conductive layer 32 and the moisture-proof barrier layer 33. Thus, the adhesive layer 36 illustrated in FIG. 5B can be eliminated. After spraying the electrically conductive layer 32 onto the moisture-proof barrier layer 33, the electrically conductive patch 35 is sewn onto the circumferential electrode 11 using an electrically conductive thread, where the thread is stitched through the electrically conductive patch 35, the moisture-proof barrier layer 33, and the electrically conductive layer 32. Alternatively, as illustrated in FIG. 8, prior to spraying the electrically conductive layer 32 onto the moisture-proof barrier layer 33, an aperture 39 can be formed in the moisture-proof barrier layer 33, the conductive patch 35 can be placed over the inner portion of the moisture-proof barrier layer 33, and the conductive layer 32 is then sprayed over both the moisture-proof barrier layer 33 and the conductive patch 35.

Next, the moisture-containing layer 31 is attached. The moisture-containing layer 31 can be formed of a material that self-adheres to the electrically conductive layer 32 and/or to the moisture-proof barrier layer 33. For example, the moisture-containing layer 31 can be formed of a layer of hydrogel, where the composition of the hydrogel on the side of the hydrogel layer adjacent to the electrically conductive layer 32 is optimized to self-adhere to the electrically conductive layer 32 and/or to the moisture-proof barrier layer 33. Thus, the moisture-containing layer 31 is placed directly on and self-adheres to the electrically conductive layer 32 and/or the moisture-proof barrier layer 33.

The composition of the hydrogel on the side of the hydrogel layer opposite the electrically conductive layer 32 (i.e., the side that contacts the patient's skin) can be optimized to self-adhere to the patient's skin and/or to the surface of the moisture-proof barrier layer 33. In some implementations, the portion of the hydrogel layer on the side opposite the electrically conductive layer 32 is formed of a laminate of 2 different compositions of hydrogel, where one of the compositions is optimized to adhere to the patient's skin, and the other composition is optimized to adhere to the surface of the moisture-proof barrier layer 33. In such implementations, layers 36, 34, and 37 of FIG. 5B can all be eliminated. Instead, a snap bracelet (not shown in FIG. 5B) is attached to the circumferential electrode in place of layer 34, in order to facilitate removal of the circumferential electrode from the patient.

As described previously, the circumferential electrode 11 can be stretchable, in order to better conform to the body part around which it is wrapped. In order for the circumferential electrode to be stretchable, the moisture-containing layer 31, the electrically conductive layer 32, the moisture-proof barrier layer 33, and the adhesive layer 36 are each formed of a material which is elastic or stretchable. Specifically, if the adhesive layer 36 is not sufficiently stretchable, the mechanical integrity of the circumferential electrode 11 may be compromised when the circumferential electrode 11 is stretched. For example, the adhesive layer 36 can be formed of 3M 6038 tape transfer stretchable adhesive.

FIG. 8 shows a cross-sectional view of another configuration for a circumferential electrode 11, illustrating the features of the electrode 11 prior to completion of assembly. As shown, in the configuration of FIG. 8, the electrically conductive patch 35 is between the moisture-proof barrier layer 33 and the electrically conductive layer 32, with the electrically conductive patch 35 directly contacting or conductively bonded to the electrically conductive layer 32. An aperture 39 is formed in the moisture-proof barrier layer 33 and the adhesive layer 36 which exposes at least a portion of the electrically conductive patch 35, the aperture 39 having a smaller cross-sectional area than that of the electrically conductive patch 35. Optionally, additional material (not shown) can be placed along the border of or around the aperture 39 on a side of the moisture-proof barrier layer 33 opposite the adhesive layer 36. The additional material, which can for example be hook and loop material, can secure additional portions of the output lead 13 to the circumferential electrode 11, and/or can provide strain relief in order to prevent the circumferential electrode 11 from becoming damaged at or near the aperture 39.

Referring still to FIG. 8, a method of manufacturing a circumferential electrode 11 is as follows. The adhesive layer 36 is first applied to the moisture-proof barrier layer 33. The adhesive layer 36 is typically electrically insulating but may be electrically conductive. Next, an aperture 39 is formed in the moisture-proof barrier layer 33 and adhesive layer 36, the aperture 39 having a smaller cross-sectional area than the electrically conductive patch 35. The electrically conductive patch 35 is then placed over the aperture 39, such that the edge of the electrically conductive patch 35 contacts the adhesive layer 36, but the central portion of the electrically conductive patch 35 is accessible through the aperture 39. As such, the edge of the electrically conductive patch is secured to the moisture-proof barrier layer 33 by adhesive layer 36.

Next, the electrically conductive layer 32 is placed over the electrically conductive patch 35 and fastened to the moisture-proof barrier layer by the adhesive layer 36. Hence, the electrically conductive layer 32 directly contacts the electrically conductive patch 35, or alternatively is secured to the electrically conductive patch 35 using a conductive adhesive material. As seen in FIGS. 5B and 6, the area of the electrically conductive layer 32 is smaller than that of both the moisture-proof barrier layer 33 and the adhesive layer 36, such that the entire area of the electrically conductive layer 32, apart from the region directly over the electrically conductive patch 35, is attached to the moisture-proof barrier layer 33 in a bulk region 51 of the circumferential electrode 11, and a border region 52 which includes only the moisture-proof barrier layer 33 and the adhesive layer 36 surrounds the bulk region 51. Next, the moisture-containing layer 31 is attached. The area of the moisture-containing layer 31 is larger than that of the electrically conductive layer 32, such that the moisture-containing layer 31 is in both the bulk and border regions. Hence, the moisture-containing layer 31 is secured to the moisture-proof barrier layer 33 by the adhesive layer 36 in the border region 52, while contacting the electrically conductive layer 32 in the bulk region 51 without any of the adhesive material being between the moisture-containing layer 31 and the electrically conductive layer 32. The border region can be wide enough to secure the layers to one another without the layers subsequently becoming detached. For example, the border region can have an average width of at least 2 mm, at least 3 mm, or at least 6 mm. Having a single adhesive layer 36, rather than also including a second adhesive layer between the moisture-containing layer 31 and the electrically conductive layer 32, simplifies the manufacturing process of the circumferential electrode 11, as well as eliminating the need for electrically conductive adhesive material within the circumferential electrode, which can be advantageous since in many cases electrically insulating adhesives may exhibit superior adhesion properties as compared to electrically conductive adhesives. The remainder of the method for forming the circumferential electrode 11 is the same as that previously described with reference to FIG. 5B.

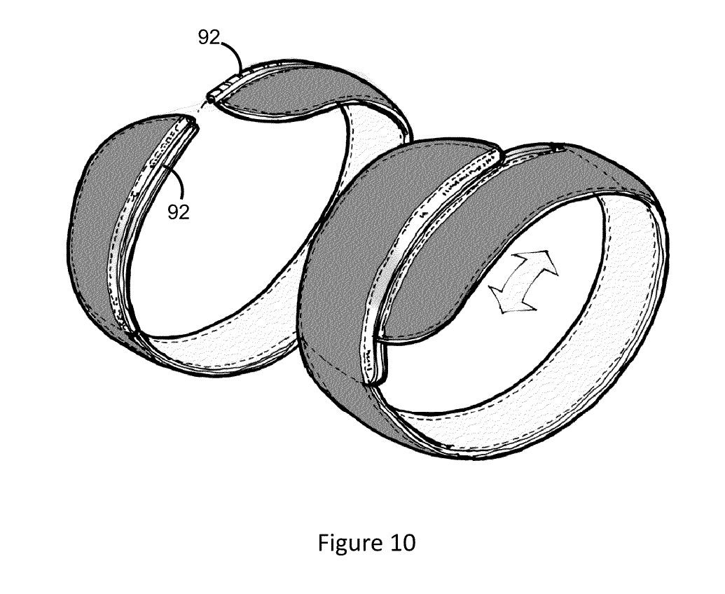

Circumferential electrodes 11 can include additional features as well, for example features provided to increase comfort level or ease of use. Examples of some of these features are shown in FIGS. 9-10. As seen in FIG. 9, a portion 91 of the hook and look fastener for securing the circumferential electrode 11 to the patient can be circular or spiral shaped, which can allow for a superior grip on the portion 91 when the circumferential electrode 11 is fastened to the patient. Or, as seen in FIG. 10, means for securing the circumferential electrode 11 to the patient can include a rail connection, which for example can include a track 92. Other features are possible as well.

A number of implementations have been described. Nevertheless, it will be understood that various modifications may be made without departing from the spirit and scope of the techniques and devices described herein. For example, in cases where the adhesive layer 36 is electrically conductive, the adhesive layer can serve as the electrically conductive layer, and so layer 32 in FIGS. 5A and 5B can be omitted. Accordingly, other implementations are within the scope of the following claims.

What is claimed is:

1. A circumferential electrode, comprising:
a moisture-containing layer comprising a first side;
an electrically conductive layer on the moisture-containing layer, the electrically conductive layer including a second side and a third side opposite the second side, the third side of the electrically conductive layer contacting the first side of the moisture-containing layer; and
a barrier layer on the electrically conductive layer, the barrier layer including a fourth side adjacent to the second side of the electrically conductive layer.

2. The circumferential electrode of claim 1, wherein the electrically conductive layer directly contacts the moisture-containing layer without any adhesive material being between the electrically conductive layer and the moisture-containing layer.

3. The circumferential electrode of claim 1, wherein the third side of the electrically conductive layer is secured to the first side of the moisture-containing layer with an electrically conductive adhesive material.

4. The circumferential electrode of claim 1, wherein the barrier layer is configured to prevent or suppress fluid escaping the moisture-containing layer.

5. The circumferential electrode of claim 1, wherein the moisture-containing layer comprises a layer of hydrogel.

6. The circumferential electrode of claim 5, wherein the layer of hydrogel includes a first portion comprising hygrogel having a first composition, wherein the first composition is configured to adhere to human skin.

7. The circumferential electrode of claim 6, the first portion further comprising hydrogel having a second composition, wherein the second composition is configured to adhere to the barrier layer.

8. The circumferential electrode of claim 7, wherein the first portion comprises a laminate including the hydrogel having the first composition and the hydrogel having the second composition.

9. The circumferential electrode of claim 6, wherein the layer of hydrogel includes a second portion comprising hygrogel having a third composition, wherein the third composition is configured to adhere to the electrically conductive layer or to the barrier layer.

10. The circumferential electrode of claim 9, wherein the second portion is on an opposite side of the layer of hydrogel from the first portion.

11. A method of forming a circumferential electrode, the method comprising:
providing a barrier layer comprising a fourth side;
attaching an electrically conductive layer comprising a second side and a third side to the barrier layer, the second side being opposite the third side, the second side being adjacent to the fourth side of the barrier layer; and
adding a hydrogel layer comprising a first side and a fifth side, the first side being opposite the fifth side, the third side of the electrically conductive layer contacting the first side of the hydrogel layer; wherein
the hydrogel layer adheres to the barrier layer or to the electrically conductive layer without requiring an additional adhesive.

12. The method of claim 11, wherein the hydrogel layer includes a first portion adjacent to the first side of the hydrogel layer and a second portion adjacent to the fifth side of the hydrogel layer, the first portion comprising hydrogel having a first composition and the second portion comprising hydrogel having a second composition, wherein the first composition is different from the second composition.

13. The method of claim 12, wherein the hydrogel in the first portion is configured to adhere to the barrier layer or to the electrically conductive layer.

14. The method of claim 13, wherein the hydrogel in the second portion is configured to adhere to human skin.

15. The method of claim 14, wherein the hydrogel in the second portion is further configured to adhere to the barrier layer.

16. A method of providing an electric current through a recipient, the method comprising:
providing a first electrode and a second electrode, the first and second electrode each contacting the recipient;
providing a current generating source connected to each of the first and second electrodes;
configuring the current generating source to provide a first electric current in a first direction through the recipient;
sensing a first voltage difference between the first electrode and the second electrode to determine a first magnitude of the first voltage difference;
comparing the first magnitude to a voltage threshold; and
executing a first function; wherein
the sensing, the comparing, and the executing are each performed by the current generating source without additional input from an operator or user of the current generating source.

17. The method of claim 16, wherein the sensing and the executing are each performed at least two times over a time span of at least one second.

18. The method of claim 16, wherein executing the first function comprises reconfiguring the current generating source to provide a second electric current in the first direction.

19. The method of claim 18, wherein the first function is executed when the first magnitude is greater than the voltage threshold.

20. The method of claim 19, the sensing and the executing each being performed at least two times over a time span of at least one second, wherein the first magnitude being greater than the voltage threshold comprises the first magnitude exceeding the voltage threshold every time the comparing is performed during the time span.

21. The method of claim 18, the first function being executed after a preprogrammed or predefined time span, wherein the first magnitude is less than the voltage threshold when the first function is executed.

22. The method of claim 18, wherein the second electric current is smaller than the first electric current.

23. The method of claim 16, further comprising executing a second function, the second function being different from the first function, wherein the second function is performed by the current generating source without requiring additional input from an operator or user of the current generating source.

24. The method of claim 23, wherein executing the first function comprises determining whether the effective resistance between the first and second electrodes is greater than a resistance threshold.

25. The method of claim 24, wherein the resistance threshold is at least 200 kilo-ohms.

26. The method of claim 23, wherein the first function is performed before the second function.

27. The method of claim 26, wherein executing the second function comprises providing an alert of an open circuit to a user or operator of the current generating source.

28. The method of claim 23, wherein executing the second function comprises reconfiguring the current generating source to provide a second electric current in the first direction.

29. The method of claim 28, wherein the second electric current is smaller than the first electric current.

30. The method of claim 28, wherein the first magnitude is greater than the voltage threshold when the second function is executed.

31. An electrotherapy system, comprising:
a first electrode and a second electrode, the first and second electrode each being configured to be connected to a biological recipient; and
a current generating source connected to each of the first and second electrodes; wherein
the current generating source is configured to provide a first electric current in a first direction through the biological recipient;
the current generating source comprises means for sensing a first voltage difference between the first electrode and the second electrode to determine a first magnitude of the first voltage difference, means for comparing the first magnitude to a voltage threshold, and means for executing a first function; and
the current generating source is operable to perform the sensing, the comparing, and the executing without additional input from an operator or user of the current generating source.

32. A method of performing electrotherapy treatments on a patient, the method comprising:
connecting a current generating source to the patient;
configuring the current generating source to provide a first electric current at a first current level setpoint through the patient, the first current level setpoint being between 500 microamps and 5 milliamps;
configuring the current generating source to provide a second electric current at a second current level setpoint through the patient, the second current level setpoint being between 10 nanoamps and 1 microamp;
passing the first current through the patient for a first time period, wherein the first current has a first mean current value over the entire span of the first time period; and
passing the second current through the patient for a second time period, wherein the second current has a second mean current value over the entire span of the second time period; wherein
the first current deviates from the first mean current value by less than 10% of the first mean current value throughout the entire first time period; and
the second current deviates from the second mean current value by less than 1% of the second mean current value throughout the entire second time period.

33. A method of performing diagnostic measurements on a patient undergoing electrotherapy, the method comprising:
providing a first electrode and a second electrode, the first and second electrodes each contacting the patient, wherein a portion of the patient's body is between the electrodes;
providing a current generating source connected to each of the first and second electrodes, the current generating source being configured to provide a current through the patient;
passing a first current through the patient for a first time period, the first current being below 200 microamps;
raising the current to a second level for a second time period, the second current level being greater than 200 microamps;
reducing the current back to the first current for a third time period; and
measuring an impedance during the second time period; wherein
the second time period is sufficiently small to prevent substantial changes in the impedance of the portion of the patient's body which is between the electrodes.

34. The method of claim 33, wherein the second time period is about 10 milliseconds or less.
